(19) United States
(12) Patent Application Publication
(10) Pub. No.: US 2002/0106407 A1
(43) Pub. Date: Aug. 8, 2002

(54) Method and apparatus for treating breakthrough pain

(76) Inventors: Dennis Coleman, Salt Lake City, UT (US); Steven A. Shoemaker, Fort Myers, FL (US)

Correspondence Address: Cephalon, Inc., 145 Brandywine Parkway, West Chester, PA, 19380, US

(21) Appl. No.: 10/013,266

(22) Filed: Dec. 10, 2001

Related U.S. Application Data
(60) Provisional application No. 60/254,627, filed on Dec. 11, 2000.

(51) Int. Cl. A61K 9/70

(52) U.S. Cl. 424/468

(57) ABSTRACT

The present invention provides methods and drug formulations comprising a drug capable of conforming to a pharmacokinetic profile when administered to a patient's systemic circulation. The pharmacokinetic profile provides a pharmacodynamic profile having an optimal onset of effect, optimal duration of effect, and an optimal rate of offset of effect. The drug formulation has a carrier for administering the drug that provides user control over the rate of absorption in order to maintain the optimal pharmacokinetic profile and the optimal pharmacodynamic profile.

### FIELD OF THE INVENTION

The present invention relates to methods and formulations for treating a patient's breakthrough pain. More specifically, the present invention relates to a pharmacokinetic (PK) curve or pharmacokinetic profile of analgesic serum concentration that results in a pharmacodynamic response (PD), pain relief, which mirrors or mimics a patient's breakthrough pain profile.

### BACKGROUND OF THE INVENTION

Pain may be generally defined as an unpleasant sensory and emotional experience associated with actual or potential tissue damage. The emotional and physiological aspects of pain are closely intertwined. Because pain is perceived by the body as an unpleasant stimulation, pain will normally evoke an emotional response. Pain may be acute, lasting days to weeks, often in response to a specific injury and often subsiding as the tissue heals. Pain may also be chronic, lasting months to years, and may persist long after initial tissue damage and healing. Chronic pain has two components, persistent pain and breakthrough pain. Persistent pain is the pain that is present most of the time, day in and day out. Breakthrough pain is a sudden flare of pain lasting minutes to hours that typically occurs several times per day on top of otherwise controlled persistent pain.

Analgesics are frequently used to treat both chronic and acute pain. Analgesics bind to receptors in the brain and spinal cord (the central nervous system or CNS) and prevent the transmission of painful stimuli to those areas of the brain that perceive pain. The pain relief effects of analgesics may also be accompanied by side effects, especially at higher doses.

The persistent component of chronic pain is typically managed by administering an analgesic on a regularly scheduled basis, so called around-the-clock (ATC) dosing. For example, a longer acting analgesic may be given every eight hours around the clock to prevent as much persistent pain as possible. The analgesic is generally supplied to the patient's blood stream at a dose that results in desired analgesic serum concentration or ASC. To be effective, this ASC should be capable of continually supplying the target tissues in the brain and spinal cord with the necessary amount of analgesic to provide a measure of pain relief, without supplying so much drug that the patient experiences intolerable side effects. Finding the right balance between pain relief and side effects is an important goal of analgesic dosing and is often difficult to achieve.

Flares of breakthrough pain occur in most patients with chronic pain, even when an appropriate dose of longer acting analgesic is being administered to effectively manage the persistent pain. These breakthrough pain episodes are often severe or excruciating, typically appear suddenly, and have a relatively short duration. An additional analgesic, above the baseline ATC analgesic, is required to manage these episodes. In order to achieve pain relief, the concentration of analgesic in the systemic circulation must be raised such that the concentration of analgesic in the target tissues, the brain and spinal cord, is high enough to block the increased pain signals reaching the pain centers in the brain. Because breakthrough pain episodes typically start suddenly, it is important the concentration of analgesic in the target tissues also rise suddenly. Finding the right balance between pain relief and side effects is just as important for managing the breakthrough pain component of chronic pain as it is for the persistent pain component. The goal for managing persistent pain is to prevent as much pain as possible. Whereas the goal for managing breakthrough pain is to get control of the pain as soon as possible after the flare begins. The analgesic used to manage breakthrough pain should also not last well beyond when the flare of pain subsides.

A popular analgesic delivery method is oral ingestion of the analgesic in the form of pills, capsules, or liquids. However, oral ingestion has several disadvantages, the foremost of which is that oral delivery is too slow in providing the target tissue with the analgesic in time to effectively treat a breakthrough pain episode. In many cases, an intravenous or other invasive procedure can supply the analgesic quickly to the systemic circulation and relatively quickly to the target tissues. However, the invasive procedures typically used to deliver analgesics often require trained medical personnel to deliver the drug. Many patients are not comfortable with invasive delivery techniques and prefer other methods. Oral transmucosal delivery is a preferred non-invasive method for delivering analgesics to patients experiencing breakthrough pain.

It is not easy to predict the right dose of an analgesic for each patient. The serum concentrations (PK profile) achieved in different individuals administered the same dose of an analgesic in the same delivery system are often quite variable. Differences in absorption, plasma protein binding, distribution, metabolism, and excretion all contribute to variability. Other sources of variability include the method of drug administration, differences in drug manufacturing, and differences in formulations. Even if the same serum and tissue concentrations are achieved, the pain relief responses (PD profiles) will vary among individuals. Responses may also vary in the same individual over time. For example, a patient's level of consciousness and emotional state can influence their perception of pain and pain relief. These numerous sources of variable responses to analgesics point to the importance of individualized dosing of analgesics, finding the right dose for each patient that provides adequate pain relief with acceptable side effects.

When managing chronic pain, the first step is often to determine the optimal dose of the ACT analgesic used to manage persistent pain. The second step is to optimize the dose of the supplemental medication used to manage breakthrough pain. Episodes of breakthrough pain may be associated with a particular event or may occur at random and be totally unpredictable. For example, breakthrough pain may occur during and after the changing of patient's wound dressings or pain may occur as the result patient activity. Other episodes may occur while a patient is sitting quietly in a chair. Episodes of breakthrough pain typically occur one to four times a day.

In order to achieve the optimal balance between pain relief and side effects, the pain relief characteristics of a supplemental analgesic should match the pain intensity characteristics of breakthrough pain. The intensity of any given episode of breakthrough pain can be described as having a profile of a quickly rising, increasing level of pain, which peaks and then subsides, with a relatively short duration. In other words, during a breakthrough pain episode, the pain stimuli received by the brain rapidly increase until peaking and then the stimuli decline. This is in contrast to persistent pain, which is present most of the time.

Oral medications typically cannot deliver analgesics to the target tissues in the CNS fast enough or at high enough concentrations to provide pain relief for many breakthrough pain episodes. Faster onset and higher analgesic serum concentrations can be reached using invasive delivery methods (such as intravenous injection) and non-invasive delivery methods such as oral transmucosal. For example, with oral transmucosal fentanyl citrate (OTFC), onset of analgesia occurs in just a few minutes, five to fifteen minutes, which is much faster than orally delivered fentanyl.

Getting more drug into the CNS faster may provide the patient with quick pain relief. However, properly treating breakthrough pain is not simply a matter of providing more drug at a faster rate. For example, administering high doses of fentanyl by rapid IV bolus injection can result in muscle rigidity, which is an unacceptable side effect outside of an inpatient, anesthesia environment. Optimal breakthrough medications should deliver analgesics rapidly to the target tissues, but not so fast that they result in unacceptable side effects.

Adverse side effects are common with patients using analgesics and particularly with potent analgesics, such as opioids. Common side effects associated with the chronic use of potent analgesics in treating breakthrough pain include: sedation, dizziness, nausea, and constipation.

Patients who experience severe breakthrough pain are sometimes willing to suffer mild adverse side effects, such as those listed above, in order to get the desired pain relief. For example, a patient in severe pain may readily tolerate a certain degree of sedation in order to achieve rapid pain relief. However, once the flare of pain has subsided, patients are much less willing to tolerate side effects. Side effects may cause patients great discomfort and become more of a concern than pain relief. And some side effects, such as muscle rigidity, are potentially life threatening. For purposes of this invention, adverse side effects that a patient experiences as a result of receiving what is substantially a minimum effective dose of an analgesic at an appropriate onset of effect and an appropriate duration of effect are referred to as “acceptable” adverse side effects. Adverse side effects that a patient experiences as a result of receiving more than the minimum effective dose of an analgesic or experiences as a result of receiving the analgesic at an inappropriate onset of effect or inappropriate duration are referred to herein as “unacceptable” adverse side effects or “unnecessary” adverse side effects. In other words, unacceptable or unnecessary adverse side effects include those adverse side effects a patient suffers that are the result of administering more analgesic than is necessary for a particular level of pain or administering the analgesic in a manner that causes analgesic serum concentration to rise too quickly or to remain high for too long.

Typical oral analgesics administered as pills, capsules and liquids have an onset of effect (pain relief) that is too slow for most breakthrough pain patients. In an attempt to achieve more rapid pain relief, the dose of these oral agents may be increased. This typically does not substantially increase the onset of pain relief but rather prolongs the analgesic effects, including side effects long after the flare of pain has subsided. If the onset of pain relief is too long, patients may also increase the dose of the longer acting ATC medication used to manage the persistent pain. This approach may also increase the frequency and severity of side effects.

Administration of the analgesic, fentanyl, by the oral transmucosal route is an example of a non-invasive manner of achieving rapid pain relief. OTFC has been shown to provide pain relief as fast as intravenous morphine. Fentanyl is an example of an opioid that moves rapidly from the blood into the brain. The pain relief effects of fentanyl can therefore be predicted from its serum concentration by accounting for the relatively short, 3-5 minute delay, for fentanyl to cross the blood-brain-barrier. FIG. 2 shows the serum PK profile for OTFC and hence the pain relief (PD) profile. This pain relief profile matches the profile of a typical episode of breakthrough pain. Cancer patients using OTFC for breakthrough pain report rapid pain relief, often within minutes, and an adequate duration of effect, but without the lingering side effects typically experienced with the oral pills, capsules, and liquids.

When patients are able to achieve rapid pain relief soon after a flare of pain first starts, they prevent the pain from achieving its maximum intensity. They no longer have to wait 20-30 minutes in severe pain for the analgesic to work. This allows them to become less focused on preventing breakthrough pain episodes. This may, for example, lower the dose of their ATC medication. Better pain control using less total analgesic means fewer analgesic related side effects.

It would be beneficial to administer analgesics using a non-invasive method and formulation that provide rapid pain relief for effectively treating breakthrough pain. Unacceptable, adverse side effects should be avoided. It would be advantageous to treat breakthrough pain with a method and formulation that promotes safety and offers unique efficacy. Patients should be able to control the balance of pain relief and side effects.

### SUMMARY OF THE INVENTION

The invention relates to methods and formulation for treating breakthrough pain. The method of the present invention reduces the likelihood that a patient will suffer unacceptable adverse side effects. More specifically, the present invention provides a method and formulation designed to produce a PK curve that results in a pain relief response (PD curve) that corresponds to, approximates, mimics, or mirrors a breakthrough pain curve.

The method of the present invention can be administered advantageously to a patient who is suffering from breakthrough pain. The method is used for a patient who is receiving a base line dose of analgesic to control an associated base line level of persistent pain, but who also has periodic episodes of acute, flare-up breakthrough pain, which require additional analgesic to bring the patient pain relief. The method may also be applied for patients who suffer from periodic painful episodes that are similar in their nature to breakthrough pain. The present invention provides the patient with a desired analgesic serum concentration that is capable of delivering substantial pain relief, while reducing unacceptable, adverse side effects, which can be associated with a patient's analgesic serum concentration. The method of the present invention comprises the steps of noninvasively delivering an analgesic at an initial absorption rate, effectuating a safe analgesic serum concentration, and providing the analgesic to the patient at a subsequent absorption rate. The method of drug delivery that results in the initial absorption rate should allow the user to change or adapt the initial absorption rate to compensate for interpatient and intrapatient variability for a given breakthrough pain episode.

The first step of the method is to deliver the analgesic to the patient's blood stream by a noninvasive delivery technique. Noninvasive delivery includes transdermal and transmucosal delivery and any other delivery routes that do not involve the puncture or incision of a patient's skin.

The analgesic is delivered noninvasively to the patient's systemic circulation at an initial absorption rate. The “initial absorption rate” is the rate at which the analgesic is absorbed into the systemic circulation during the period in which the analgesic serum concentration in the patient's systemic circulation is rising or increasing. The initial absorption rate may differ from one patient to another and one administration to another depending upon the needs of the patient and the administration method used. The initial absorption rate increases analgesic serum concentration in a manner that reduces the potential for unnecessary adverse side effects that are associated with excessively rapid increases in analgesic serum concentration.

The initial absorption rate produces a clinically beneficial analgesic serum concentration during the period of time in which the analgesic serum concentration is increasing in the patient's systemic circulation. A clinically beneficial ASC provides an analgesic level that promotes the onset of meaningful therapeutic relief during a breakthrough pain episode. In other words, the analgesic serum concentration must be high enough for the analgesic to reach the target tissues in the CNS at a rate and levels sufficient to give the patient significant pain relief. The onset of pain relief should come quickly. If the analgesic acts too slowly, the patient may suffer too long in pain and/or the episode will pass before the drug takes effect.

Another step in the present invention is to effectuate a safe ASC. As the ASC in the patient's blood stream increases, so does the risk of overmedication. Overmedication may lead to discomfort and suffering from unnecessary side effects that have the potential to become serious and life threatening. To establish a safe ASC, the administration of the drug and associated absorption rate should be monitored and adjusted to cause analgesic serum concentration to peak in a timely fashion. The safe ASC, which reduces the potential for overdosing and unnecessary adverse side effects, must also be capable of managing the patient's breakthrough pain by supplying the target tissues with a sufficient amount of drug to reduce and substantially eliminate the pain experienced during a breakthrough pain episode.

Having delivered the analgesic to a patient's circulation system at an initial absorption rate to increase the ASC and provide rapid pain relief, the analgesic may be provided to the patient at a subsequent absorption rate. The subsequent absorption rate will provide an adequate duration of pain relief and will allow the ASC to decrease as the analgesic is eliminated from the circulation. Like the initial absorption rate, the subsequent absorption rate produces a clinically beneficial ASC. Thus, the clinically beneficial decreasing ASC continues to promote substantial therapeutic pain relief.

The subsequent absorption rate and elimination of the analgesic from the circulation must also reduce the potential for unnecessary adverse side effects associated with a lingering, elevated ASC. The subsequent absorption rate reduces the likelihood of excessive dosing during the ASC decrease period. Moreover, the subsequent absorption rate may also reduce the potential for unnecessary adverse side effects associated with an elevated ASC at a time when the intensity of the breakthrough pain episode has subsided. The subsequent absorption rate balances the need for a clinically beneficial ASC to provide an adequate duration of pain relief with the need to eliminate the analgesic from the circulation once the pain episode has subsided. The initial absorption rate and subsequent absorption rate are the result of drugs delivered using a delivery technique, which reduces secondary absorption of analgesic, such as from depot sites or inadvertently ingested drugs.

The present invention also relates to a drug formulation. The drug formulation of the present invention comprises an analgesic that is noninvasively administered to a patient's systemic circulation. The analgesic delivery is capable of conforming to a pharmacokinetic profile. The pharmacokinetic profile represents the analgesic serum concentration in the patient's systemic circulation over time. The analgesic in the systemic circulation is absorbed into a target tissue (i.e. brain or spinal cord) in effective amounts. The effect of the drug on the target tissue results in a pharmacodynamic profile. In the present invention, the pharmacodynamic profile has a substantially optimal rate of onset of effect, a substantially optimal duration of effect, and a substantially optimal offset of effect. The PD profile substantially mirrors, mimics, or corresponds to a patient's breakthrough pain profile.

The PK profile of the present invention that results in a PD profile having a substantially optimal onset of effect allows target tissues to be supplied with an analgesic in amounts that give timely and substantial therapeutic relief for patients experiencing a breakthrough pain episode. At the same time, the PK profile maintains an analgesic serum concentration within a range that reduces the potential for unnecessary adverse side effects associated with rapid increases in ASC. The PK profile allows analgesic to be provided to target tissues for a period of time that is substantially limited to the duration of the breakthrough pain episode and does not extend long after the episode has ended. Additionally, the PK profile allows analgesic to be supplied to target tissues in amounts that manage pain during the offset of a breakthrough pain episode. The amount and rate of analgesic also reduces the potential for unnecessary adverse side effects associated with a lingering, elevated ASC.

The drug delivery system in the present invention provides effective user control over the rate of absorption. The effective user control allows the user to administer the drug in a manner that provides a PK profile that produces the PD profile described above. Thus, the delivery system provides the user control over the rate of absorption to maintain an optimal pharmacokinetic profile that results in an optimal pharmacodynamic profile.

When using prior art formulations and methods, interpatient and intrapatient variability make it difficult to determine a safe and effective dose that optimally balances pain relief and side effect for individual patients. The PK and PD profiles of prior art techniques do not effectively mirror or mimic a patient's specific breakthrough pain profile curve. The present invention provides a pharmacodynamic pain relief response that corresponds to, approximates, mimics, or mirrors a patient's breakthrough pain curve. The pharmacokinetic curve of the present invention results in pharmacodynamic response that can mirror or correspond to a breakthrough pain profile as explained below. The PK curve can be adjusted during administration to take into account variability in a patient's pharmacokinetic and pharmacodynamic profiles. The PK profile shows an increase in ASC as the user begins the administration of the drug. The administration may begin when the patient begins to feel significant pain above the base line persistent pain, that is, when the breakthrough pain has crossed the patient's threshold for baseline pain. The rate of increase in the analgesic serum concentration may be adjusted by the user to provide a pain relief response that approximates the rate of increase in the intensity of the breakthrough pain. The positive slope of the PK curve may decrease as a result of the user adjusting the rate of absorption when analgesic concentration in systemic circulation is sufficient to affect the target tissue and thereby reduce the patient's pain. The PK curve may exhibit another decrease in the positive slope as the result of the user significantly reducing or terminating absorption of the analgesic at a point sometime after the patient's perceived pain begins to be reduced but before the patient's pain is completely relieved or eliminated. The PK profile results in an ASC providing a duration of pain relief long enough to manage the episode of breakthrough pain. The PK curve has a negative slope, which is the result of terminating administration of the drug, distribution of drug into the tissues, metabolism and excretion of the drug in a manner that is not significantly complicated by delayed absorption from depot sites or secondary absorption. Thus, the PK profile and resultant PD pain relief profile of the present invention substantially mimics or mirrors the breakthrough pain profile.

In accordance with the invention broadly described above, it is an object of at least one embodiment of the present invention to reduce unacceptable adverse side effects associated with treating breakthrough pain with analgesics.

It is an another object of at least one embodiment of the present invention to reduce unacceptable adverse side effects associated with excessively rapid increases in ASC.

It is another object of at least one embodiment of the present invention to reduce unacceptable adverse side effects associated with excessively elevated ASC.

It is another object of at least one embodiment of the present invention to reduce unacceptable adverse side effects associated with lingering, elevated ASC.

It is another object of at least one embodiment of the present invention to provide a safe and effective ASC.

It is another object of at least one embodiment of the present invention to provide analgesic to a patient's systemic circulation at a rate that effectively manages breakthrough pain.

It is another object of at least one embodiment of the present invention to use both the patient's awareness of pharmacodynamic factors of analgesic administration and an understanding of the pharmacokinetic factors involved in analgesic administration to produce a treatment, which provides a substantially minimum effective dose of analgesic for the patient's breakthrough pain.

It is another object of at least one embodiment of the present invention to provide a PD and PK based approach to administering analgesic for breakthrough pain.

It is another object of the present invention to provide a method and formulation that yield a PK curve having safety with unique efficacy.

It is another object of at least one embodiment of the present invention to provide the patient better control over the balance of pain relief and side effects associated with the use of analgesics.

Additional objects and advantages of the invention will be set forth in the description that follows, and in part will be obvious from the description, or may be learned by the practice of the invention. The objects and advantages of the invention may be realized and obtained by means of the instruments and combinations particularly pointed out in the appended claims.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a breakthrough pain profile with a corresponding minimum effective dose profile; that is, the graph shows hypothetical minimum effective dose covering the pain and corresponding hypothetical minimum effective dose;

FIG. 2 shows the PK profile of an OTFC unit delivered using a prior art method. The analgesic effects of fentanyl are related to the serum concentration if proper allowance is made for delay into an out of the CNS (a process with a relatively short three to five minute half-life);

FIG. 3 shows a hypothetical PK curve of analgesic serum concentration that represents an analgesic dose that is lower than necessary for therapeutic pain relief, that is, the graph shows insufficient ASC for the given hypothetical pain intensity and corresponding hypothetical minimum effective dose;

FIG. 4 shows a hypothetical PK curve of analgesic serum concentration that represents an analgesic dose that is higher than necessary for therapeutic pain relief, that is, the graph shows excessive ASC for the given hypothetical pain intensity and corresponding hypothetical minimum effective dose;

FIG. 5 shows a hypothetical PK curve of analgesic serum concentration with an undesirably fast rate of increase in ASC superimposed upon a hypothetical breakthrough pain profile, that is, the graph shows excessively fast increase in ASC for the given hypothetical pain intensity and corresponding hypothetical minimum effective dose;

FIG. 6 shows a hypothetical PK curve of analgesic serum concentration that is superimposed upon a hypothetical breakthrough pain profile, that is, the graph shows an a undesirably slow rate of increase in analgesic serum concentration;

FIG. 7 shows a hypothetical PK curve of analgesic serum concentration with a rate of decrease in analgesic serum concentration that is undesirably slow superimposed upon a hypothetical breakthrough pain profile, that is the graph shows a lingering, elevated ASC for a given hypothetical pain intensity and corresponding hypothetical minimum effective dose;

FIG. 8 shows a hypothetical PK profile of analgesic serum concentration with a rate of decrease in analgesic serum concentration that is undesirably fast superimposed upon a hypothetical breakthrough pain profile, that is, the graph shows an excessively rapid decrease in ASC for a given hypothetical pain intensity and corresponding hypothetical minimum effective dose;

FIG. 9 shows a hypothetical PK curve of analgesic serum concentration, which mirrors or mimics a superimposed hypothetical breakthrough pain curve, that is, the graph shows hypothetical pain intensity and a corresponding hypothetical PK curve;

FIG. 10 shows a hypothetical PK curve of analgesic serum concentration, which mirrors the minimum effective dose of a patient's breakthrough pain, that is, the graph shows hypothetical pain intensity and a corresponding hypothetical minimum effective dose with a corresponding PK curve; and

FIG. 11 shows a hypothetical PK curve of analgesic serum concentration, which substantially mirrors the minimum effective dose of a patient's breakthrough pain, that is, the graph shows hypothetical pain intensity and a corresponding hypothetical minimum effective dose with a substantially corresponding PK curve.

### DETAILED DESCRIPTION OF THE PREFERRED EMBODIMENTS

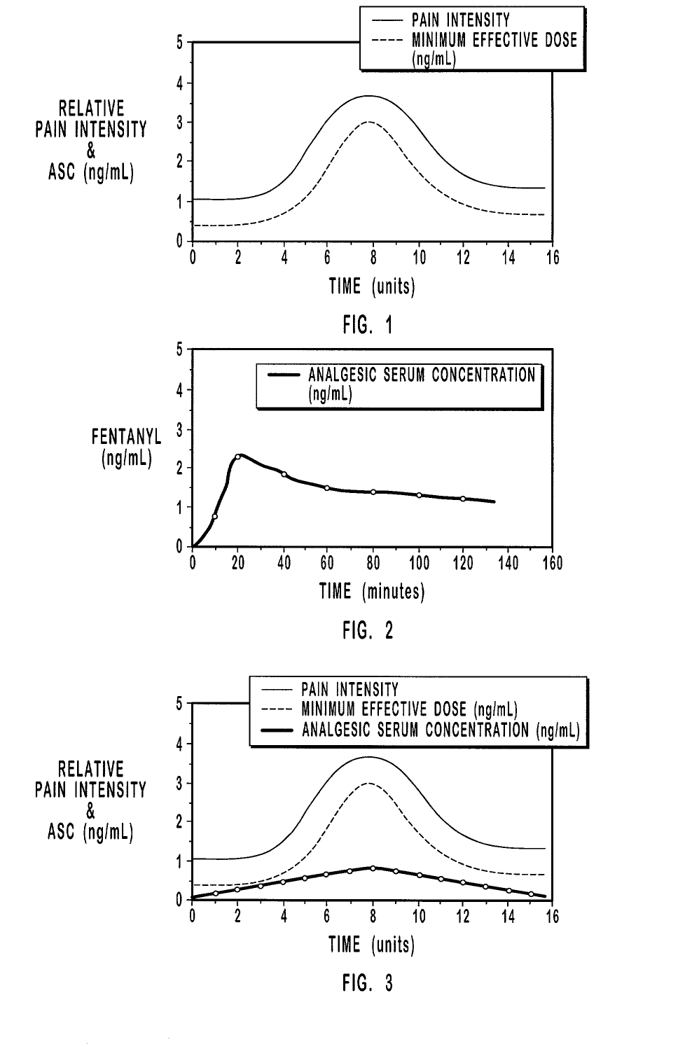

It will be readily understood that the components of the present invention, as generally described and illustrated in the figures herein, could be arranged and designed in a wide variety of different configurations. Thus, the following more detailed description of the embodiments of the system and method of the present invention, as represented in FIGS. 1 through 7, is not intended to limit the scope of the invention, as claimed, but is merely representative of the presently preferred embodiments of the invention.

The presently preferred embodiments of the invention will be best understood by reference to the drawings.

FIG. 1 shows a hypothetical breakthrough pain profile (as represented by a hypothetical minimum effective dose) with a corresponding hypothetical minimum effective dose. As a patient experiences breakthrough pain, the appropriate minimum effective dose rises and falls corresponding to the pain level that the patient is experiencing. Therefore, the minimum effective dose has a profile that, when plotted, corresponds to or mimics the breakthrough pain episode. The profile of the minimum effective dose is therefore affected by the variability in the breakthrough pain episode as experienced by the patient as well as the variability between patients to the effects of a given analgesic. The minimum effective dose therefore is affected both by pharmacodynamic (PD) and pharmacokinetic (PK) variability. Unfortunately, noninvasive prior art methods of treating breakthrough pain are not designed or administered to produce a pharmacokinetic profile that mirrors or mimics the breakthrough pain profile. In other words, non-invasive prior art techniques deliver the analgesic in a way that does not correspond to or that fails to mirror the pharmacodynamic profile necessary to manage the breakthrough pain episode and/or the minimum effective dose profile.

FIG. 3 shows a hypothetical PK curve for delivery of an analgesic for which the ASC is insufficient to control breakthrough pain. The curve of FIG. 3 indicates that the analgesic serum concentration has not reached the necessary minimum effective dose level to offer the patient (meaningful) pain relief. Prior art analgesic administration techniques can produce curves similar to the curve in FIG. 3 where a significant amount of the analgesic is eliminated or cleared before it reaches the systemic circulation. For example, a large percentage of oral analgesics are eliminated by the first pass effect. To be effective as a treatment for breakthrough pain, the analgesic delivery method must provide sufficient concentrations of analgesic to the blood stream and thereby to the target tissue (the CNS) to bind to pain receptors and thereby significantly reduce the patient's pain.

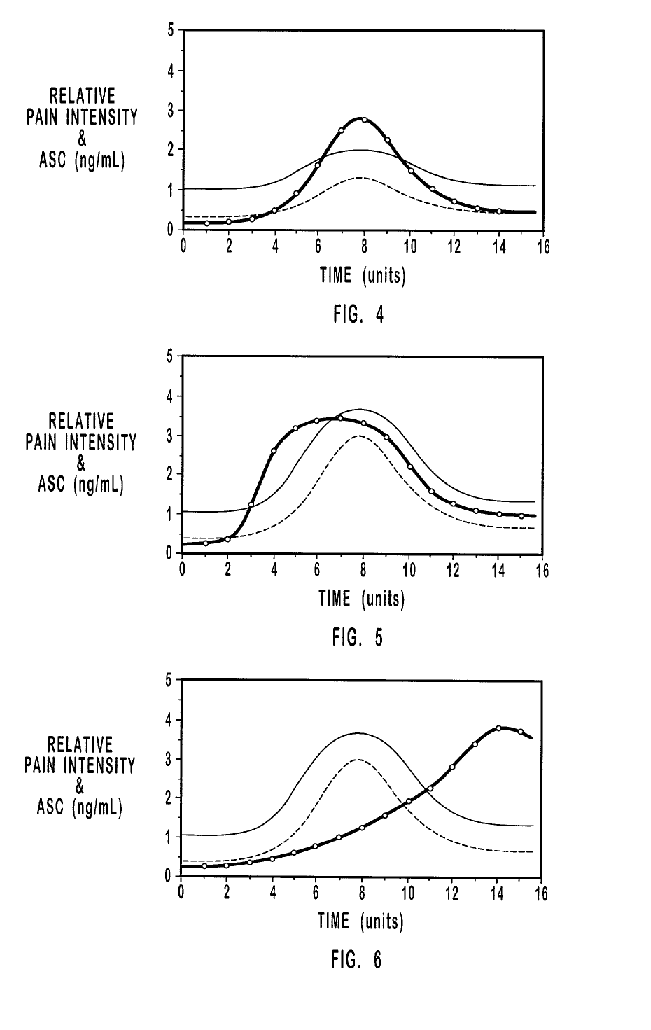

FIG. 4 illustrates a PK profile of an analgesic serum concentration that is unnecessarily high and therefore increases risk to the patient of unnecessary adverse side effects. The PK curve shows that the analgesic serum concentration is far above the level necessary to treat the patient's pain. Patients may be willing to suffer many adverse side effects in order to get pain relief and therefore may be willing to accept high doses of analgesic that increase and often result in those adverse side effects. However, an analgesic serum concentration that is significantly higher than is necessary to manage a patient's breakthrough pain exposes the patient to unnecessary adverse side effects and offers no reciprocal benefit of increased pain relief.

An example of a profile with an excessively high analgesic concentration can be seen in the use of bolus injections of analgesic administered to the systemic circulation. Because the pattern of each breakthrough pain episode experienced by a patient is variable and because the patient's response to the analgesic can vary, it is difficult to know at the time of administration whether the dosage is going to be excessive for the given pain episode. If the dose is excessive, the patient may suffer from unnecessary adverse side effects. Rather than injecting a large bolus, one embodiment of the present invention uses a method in which the dosage is administered in a non-invasive manner in very small portions over a period of time or at a continuous, controllable rate. This type of administration reduces the chances of the patient receiving a significantly greater dose than is necessary to treat the pain. If the dose is administered in small portions, the user can evaluate the progressive effect of the analgesic and terminate the administration at an appropriate time to avoid overmedication.

FIG. 5 shows a PK profile having a rate of increase in analgesic serum concentration that is excessively rapid. Excessively rapid increases in opioid analgesics are associated with serious adverse side effects, such as muscle rigidity. This dangerous side effect underscores the need for an improved analgesic delivery method that allows the effect of the analgesic to be rapid, but not too rapid. Techniques which give the patient large or substantial doses of analgesic rather than smaller, consecutive doses increase the chances the patient will suffer from an excessively rapid increase in analgesic serum concentration and thereby suffer a dangerous, unacceptable adverse side effect. Such rapid increases may also result in a euphoric feeling in the patient, which can lead to patient abuse of the drug.

FIG. 6 shows a relatively slow rate of increase in analgesic serum concentration such that the peak of the analgesic serum concentration is not timely relative to the peak of the breakthrough pain episode. In other words, the level of analgesic serum concentration does not rise quickly enough to keep pace with the increased pain intensity of the breakthrough episode. The slow rise in analgesic serum concentration results in two significant disadvantages. First, the analgesic does not arrive in time at the target tissue and does not arrive in sufficient concentration to treat the breakthrough pain, and therefore the patient unnecessarily suffers pain. Second, the serum concentration peaks and remains elevated after the peak in the breakthrough pain episode, the patient is exposed to the adverse side effects that accompany high levels of analgesic, but does not receive any benefit from such exposure. In other words, the patient suffers from the pain of the breakthrough episode and then, as analgesic serum concentration rises, is subsequently exposed to the adverse side effects associated with an elevated analgesic serum concentration.

Prior art non-invasive analgesic administration techniques result in a PK profile having an unacceptably slow increase in ASC when the administration technique does not take into account the delay caused by the particular absorption rate of analgesic into the systemic circulation from the administration site. This slow absorption into the systemic circulation results in a delay in the delivery of the analgesic to the target tissue. In order to compensate for the slow onset of effect, patients may try higher doses of prior art analgesics. This usually does not increase the onset of effect, but does increase the risk of unnecessary side effects from elevated lingering ASC once the pain has subsided. The present invention employs analgesic delivery techniques which do not result in substantially delayed increases in analgesic serum concentration and thus prevent the delayed peak and accompanying discomfort and unacceptable adverse side effects.

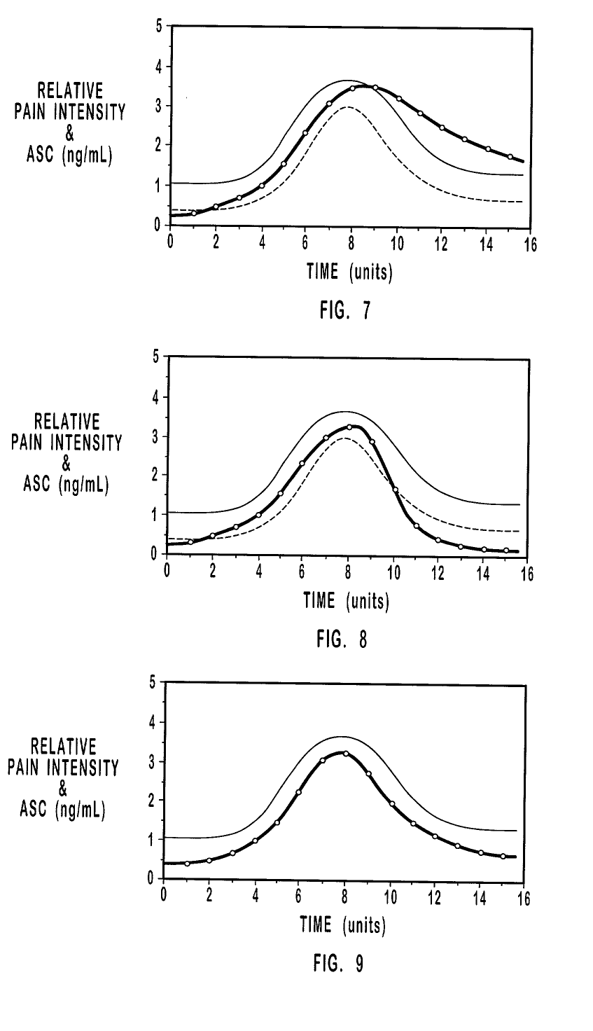

FIG. 7 illustrates a PK profile having a rate of decrease in analgesic serum concentration that is unnecessarily slow. In such a profile, the analgesic serum concentration remains higher than is necessary to control the breakthrough pain episode as the breakthrough pain episode subsides, which exposes the patient to elevated, lingering ASC. Exposing the patient to these elevated, lingering analgesic serum concentrations increases the likelihood that the patient will suffer from unnecessary adverse side effects. The elevated levels create “a tail” or “shadow” on the PK profile. The tail may result from an analgesic delivery technique that does not take into account the time it takes for the analgesic to reach the target tissue from the administration site or does not account for secondary absorption.

FIG. 8 shows a PK curve with an excessively fast rate of decrease in analgesic serum concentration. In situations in which the excessively fast rate of decrease in analgesic serum concentration corresponds, there may not be enough analgesic at the pain receptors in the target tissue. The duration of pain relief will not be long enough to manage the breakthrough pain episode, and the patient will suffer.

FIG. 9 shows an advantageous PK profile for an analgesic, such as fentanyl, that rapidly moves from the blood in and out of the CNS. The pain relief response mirrors or mimics the breakthrough pain profile closely so as to avoid unnecessary adverse side effects and yet provides the patient with sufficient pain relief from the breakthrough pain episode. For example, the PK profile may be the result of an analgesic administration method that yields an analgesic serum concentration in the blood stream and subsequently delivers sufficient analgesic to the target tissue to provide the patient with relief from the breakthrough pain episode. However, the method does not provide an excessively high dose of analgesic to the systemic circulation, and so does not unnecessarily increase the patient's exposure to unacceptable adverse side effects.

The PK profile of FIG. 9 also provides pain relief to the patient in a timely fashion by increasing the analgesic serum concentration at a rate that mimics or mirrors the rise in intensity of the pain in the breakthrough pain episode. Timely delivery of the analgesic reduces the likelihood the patient will suffer unnecessarily from the breakthrough pain episode and reduces the likelihood that the patient will be exposed to unacceptable adverse side effects. Additionally, an administration technique and/or formulation that produces a PK profile like that shown in FIG. 9 will also avoid the dangers of excessively rapid increases in analgesic serum concentration.

Significantly, the PK profile of FIG. 9 shows an analgesic serum concentration that does not linger or remain elevated after the breakthrough pain episode has subsided. Analgesic serum concentrations that mimic or mirror the breakthrough pain episode as the episode subsides reduce the likelihood that the patient will suffer from unnecessary adverse side effects associated with lingering, elevated ASC. Likewise, if a patient's analgesic serum concentration mirrors the breakthrough pain curve as the breakthrough pain episode subsides, it reduces the chance that the analgesia will wear off before the breakthrough pain episode is concluded.

The present invention provides a PK curve having an upward slope that mimics the upward slope of a patient's specific breakthrough pain profile. The methods and formulation of the present invention reduce the likelihood that the analgesic serum concentration, represented by the PK curve, will increase too quickly. The present invention reduces the likelihood that the analgesic will reach the target tissue in high doses that cause unacceptable adverse side effects. For example, one embodiment of the present invention supplies the analgesic in small repetitive doses, thereby allowing the user to control the amount of analgesic that enters the system and terminate the absorption at an appropriate level of analgesic. Another embodiment of the present invention may use a formulation having a time release or controlled release formulation of the analgesic, which may prevent the analgesic from reaching the target tissue in excessively high concentrations.

The methods and formulations producing the PK curve of the present invention also decrease the likelihood that the analgesic serum concentration will increase at a rate that is too slow. One embodiment of the present invention provides for administering the analgesic at an administration site that is “closer” to the target tissue so that the analgesic takes less time to reach the target tissue and travels more directly to the target tissue. Another embodiment of the present invention may provide a drug formulation and delivery method that increases the speed of absorption into the systemic circulation and/or target tissue.

The present invention also provides a PK curve that mirrors the peak of a patient's specific breakthrough pain curve. Methods and formulations of the present invention reduce the likelihood that the analgesic serum concentration will be delivered to the systemic circulation at an excessively high dose. This will reduce the likelihood that too much analgesic will be absorbed into the target tissue. For example, one embodiment of the present invention may employ a form of user control. The delivery method allows the user to progressively or continuously evaluate the analgesic effect in order to determine when the effect of the analgesic is sufficient for the patient's pain and when administration of the analgesic should be modified or terminated. User control then allows the user to make the necessary modifications in administration.

Methods and formulations of the present invention also reduce the likelihood that an ineffectively low dose of the analgesic will be administered. In order to ensure that enough analgesic reaches the target tissue, one embodiment of the present invention may provide a formulation that increases absorption of an analgesic into the systemic circulation and to the target tissue. Likewise, another embodiment of the present invention may increase the release of the analgesic from its dosage form, making more analgesic available for absorption into the systemic circulation.

The present invention also provides a PK curve that mirrors the downward slope of a patient's specific breakthrough pain curve. Methods and formulations of the present invention reduce the likelihood that the decrease in analgesic serum concentration in a patient's systemic circulation and/or target tissues will fall too quickly. The present invention maintains sufficient delivery of analgesic to the systemic circulation to prevent an unacceptably rapid rate of decrease in analgesic serum concentration during the period of time when the breakthrough pain episode subsides. One embodiment of the present invention provides a method for maintaining sufficient delivery of analgesic by allowing the user to control the amount by which the dose is reduced as the pain subsides. Another embodiment of the present invention provides a method for increasing the analgesic serum concentration level until a specific pharmacodynamic effect is achieved. For example, the ASC may be increased until the increase in pain begins to subside. This alternative embodiment will reduce the likelihood the ASC will drop too fast or too soon.

The methods and formulations of the present invention also reduce the likelihood that the analgesic serum concentration in the systemic circulation and target tissues will decrease too slowly. A slow rate of decrease may result in the patient experiencing unacceptable adverse side effects from elevated, lingering associated serum concentration. One embodiment of the present invention provides a method in which the administration is terminated at a time that takes into account any delayed absorption of the analgesic and thereby reduces the chance of an unacceptable elevated, lingering ASC. Another embodiment of the present invention reduces the likelihood of analgesic being absorbed into the systemic circulation through secondary absorption routes, such as when an analgesic being delivered oral transmucosally is instead ingested, or when significant amounts of analgesic are absorbed into secondary tissues, creating depot sites. This alternative embodiment also reduces the likelihood of unacceptable adverse side effects.

The present invention provides a PK profile that provides safety with unique efficacy. The PK profile of the present invention can be substantially adapted during administration to account for interpatient and intrapatient variability. In doing so, the methods and formulations that produce the PK profile of the present invention provide a more effective treatment. In some embodiments of the present invention, some of the causes of variability, such as variability due to differences in user administration, are utilized as a means to adapt or modify the treatment to meet the client's specific needs. By employing factors that create variability to tailor the delivery of the drug to the patient's specific needs, the present invention turns what is often perceived as a disadvantage into an advantage in effective treatment.

The PK profile of the present invention provides for an initial increase in analgesic serum concentration. This initial increase occurs as the result of administering additional analgesic into the patient's systemic circulation and thereby to the target tissue at the beginning of a breakthrough pain episode. The PK profile also provides for a rate of increase in the analgesic serum concentration that is adjusted or tailored to the patient's perception of increasing breakthrough pain. A decrease rate of rise in the ASC is provided in a timely manner as a result of the user slowing the administration of the drug as the analgesic begins to take effect. The peak in the ASC occurs as the rate at which the drug is absorbed into the patient's systemic circulation begins to lag behind the rate at which the analgesic is eliminated from the patient's system.

In one embodiment of the present invention, the administration of the analgesic is terminated before the perceived pain is completely eliminated or alternatively before the breakthrough pain peaks. The PK profile of the present invention has a rate of decrease in an analgesic serum concentration that is not affected by delayed analgesic absorption from secondary absorption routes. In one embodiment, an analgesic formulation is specifically designed to prevent absorption of an analgesic through the GI tract. Similarly, in another embodiment, the method of administration is designed to prevent secondary absorption of the analgesic from the GI tract. In another embodiment, the method is designed to prevent untimely absorption of analgesic from depot sites.

In another embodiment of the present invention, a lozenge containing an analgesic, which can be delivered oral transmucosally, is administered to a patient for treating a breakthrough pain episode. The patient's PK profile of the analgesic mirrors or mimics the breakthrough pain curve. As the pain increases, the patient sucks more vigorously on the lozenge to speed the rate of increase in analgesic serum concentration. Later, as the rate at which the increase in pain begins to subside, the patient sucks less vigorously on the lozenge to decrease the speed at which the analgesic serum concentration rises. The analgesic continues to be administered according to the patient's specific pain level until the patient receives substantial pain relief. The administration can be terminated by removing the lozenge. During administration, the patient may reduce the absorption rate before the breakthrough pain is completely eliminated and may terminate the administration before the breakthrough pain episode completely subsides in order to account for any delay in absorption into the target tissues from the systemic circulation. The patient may also expectorate any excess saliva mixed with analgesic formulation in order to prevent ingestion and subsequent secondary absorption.

An alternative embodiment of the present invention employs a lozenge having a drug formulation, which includes an analgesic and a carrier. The carrier may reduce GI absorption of any analgesic that is swallowed during the administration. The carrier may also increase absorption of analgesic through the oral mucosa.

In another example, a nasal spray containing an analgesic for treating breakthrough pain is administered to a patient. The analgesic is delivered through the nasal mucosa in small doses. Delivery to the nasal mucosa is accomplished in a manner that minimizes absorption of the analgesic into secondary tissues through the nasal passage. As the breakthrough pain increases, the small doses of the analgesic are applied more often. The user adjusts the number of applications and the amount of each dose in the application according to the increase in the breakthrough pain. The user continues administering the analgesic according to the patient's specific pain level and provides sufficient analgesic to give substantial pain relief. Application of the nasal spray may be reduced before the breakthrough pain is completely eliminated and is terminated before the breakthrough pain subsides completely in order to allow for any time delay in the absorption of the analgesic into the target tissues from the systemic circulation. The nasal spray may be administered in a fine mist, atomized or aerosol form, or may be employed in a form of bioadhesive in order to reduce the chance of the formulation being ingested. Similarly, the patient may be instructed to spit out or expectorate any excess formulation that is carried down into the esophagus and toward the stomach.

In an alternative embodiment, the nasal spray may have a formulation comprising a drug and a carrier. The carrier may reduce gastrointestinal absorption of the analgesic or increase absorption of the analgesic in the nasal and surrounding mucosa.

In another embodiment of the present invention, a patient experiencing breakthrough pain is treated with an analgesic delivered through a buccal patch. The user may remove and reapply the patch as needed to adjust the absorption rate of the analgesic or the release rate of the drug may be controlled and adjusted by methods known in the art. Alternatively, in order to control the absorption rate of the analgesic, the patch itself may be configured so that the surface area of oral mucosa exposed to the analgesic formulation in the patch is limited or expanded. The analgesic is administered in this fashion according to the patient's specific pain level. Administration may be reduced before the breakthrough pain is completely eliminated, and is terminated some time before the breakthrough episode completely subsides in order to account for any delay in absorption into the target tissue as a result of the oral transmucosal delivery. The portion of the oral mucosa that contacts the analgesic formulation can be limited in order to limit the potential for depot sites in the mucosa.

Alternatively, the buccal patch may have an adjustable rate limiting membrane. The rate at which a drug crosses the membrane may be affected by the pressure on or around the membrane.

Another embodiment of the present invention provides the PK profile that is safe and uniquely effective. A patient is treated for breakthrough pain by sucking on a lozenge attached to a holder designed for oral transmucosal delivery. Administering the lozenge by manipulating the attached holder causes an initial increase in analgesic serum concentration at the beginning of the breakthrough pain episode. By sucking on the lozenge more or less vigorously and by removing the lozenge from the mouth, the patient is able to adjust the rate of increase in the analgesic serum concentration to match the patient's perception of the increasing pain. As the analgesic begins to take effect, the patient can decrease the analgesic absorption rate. The patient control thereby reduces the likelihood of an overdose or underdose of analgesic and allows the peak analgesic serum concentration to be safe and effective. The patient is instructed to terminate the administration of analgesic sometime before the actual breakthrough pain episode has completely subsided. To do so, the patient removes the lozenge from his or her mouth using the handle attached to the lozenge. In order to prevent any delayed absorption of analgesic through secondary absorption routes, the patient is instructed to minimize swallowing of the analgesic formulation. The patient may be instructed to expectorate the formulation if necessary. Alternatively, the lozenge attached to a handle comprises an analgesic formulation having a carrier that reduces absorption of the analgesic through the GI tract.

In yet another embodiment of the present invention, a patient experiencing breakthrough pain is treated with an analgesic delivered through a transdermal patch. The patient may apply, remove, and reapply the patch as needed to adjust the absorption rate of the analgesic to match the breakthrough pain profile. Alternatively, the patch may be configured to release the analgesic at an adjustable rate, using methods known in the art.

In another embodiment, a patient is treated with an analgesic delivered through an oral spray.

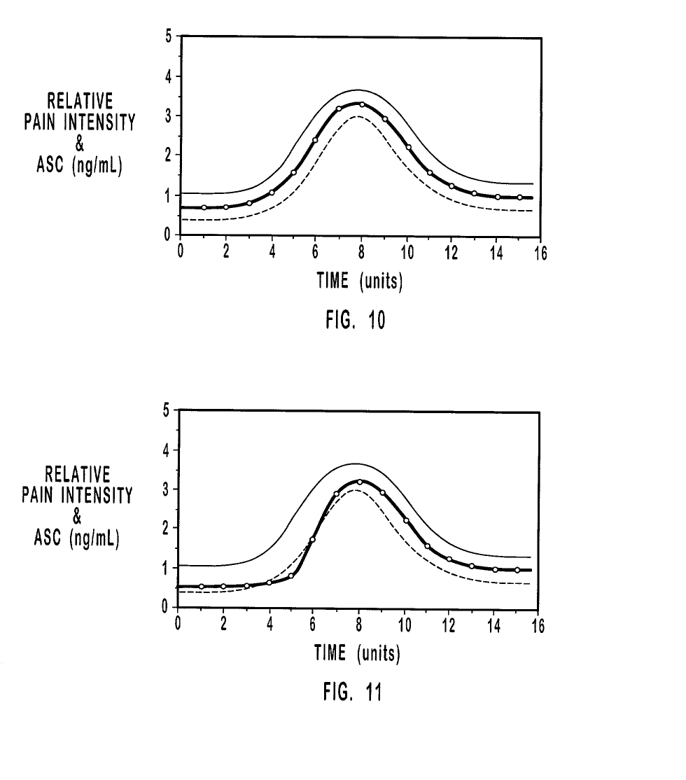

FIG. 10 shows a hypothetical pharmacokinetic curve of an analgesic serum concentration with a hypothetical corresponding minimum effective dose curve. The profile of the minimum effective dose curve is dependent upon the specific breakthrough pain episode a patient experiences. To provide effective relief to the patient for the pain, the embodiment of FIG. 10 shows the pharmacokinetic curve following the minimum effective dose profile. It is understood that in practice the PK profile should substantially correspond or mimic the minimum effective dose profile and the breakthrough pain curve, as shown in FIG. 11. The PK profile may be slightly above or slightly below and/or slightly ahead of or behind the minimum effective dose, but must provide the patient with meaningful therapeutic relief from the pain. Preferably, the PK profile yields an analgesic serum concentration that provides the target tissue with precisely the minimum effective dose or an amount of analgesic just slightly above the minimum effective dose. The present invention is a dose level that takes into account the changing levels of medication that are required to provide the patient with relief from the breakthrough pain episode.

Potential drugs for use with the present invention include, but are not limited to: morphine, hydromorphone, levorphanol, heroin, fentanyl, sufentanil, alfentanil, remifentanil, fentanyl derivatives, methadone, buprenorphine, and oxycodone.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

What is claimed is:

1. A method for reducing unnecessary adverse side effects associated with a patient's analgesic serum concentration (ASC) when the patient is being treated for breakthrough pain, the method comprising the steps of:
i) non-invasively delivering an analgesic into a patient's systemic circulation at an initial absorption rate, said initial absorption rate producing a clinically beneficial, increasing ASC, said initial absorption rate reducing the potential for an unnecessary adverse side effect associated with excessively rapid increases in ASC, said clinically beneficial, increasing ASC promoting an onset of meaningful therapeutic relief during a breakthrough pain episode;
ii) effectuating a safe, ASC, said safe ASC capable of managing the patient's breakthrough pain; and
iii) providing the analgesic to the patient's systemic circulation at a subsequent absorption rate, said subsequent absorption rate providing a clinically beneficial decreasing ASC, said subsequent absorption rate reducing the potential for an unnecessary adverse side effect associated with a lingering, elevated ASC and said subsequent absorption rate reducing the potential for an unnecessary adverse side effect associated with an excessively rapid decrease in the patient's ASC.

2. The method of claim 1, wherein the step of non-invasively delivering an analgesic into a patient's systemic circulation comprises delivering the analgesic transmucosally.

3. The method of claim 1, wherein the step of non-invasively delivering an analgesic into a patient's systemic circulation comprises delivering the analgesic transdermally.

4. The method of claim 1, wherein the step of non-invasively delivering an analgesic into a patient's systemic circulation comprises delivering the analgesic through the nasal mucosa.

5. The method of claim 1, wherein the step of non-invasively delivering an analgesic into a patient's systemic circulation comprises delivering the analgesic with an oral spray.

6. The method of claim 1, wherein the step of non-invasively delivering an analgesic into a patient's systemic circulation comprises delivering the analgesic with a nasal spray.

7. The method of claim 1, wherein the step of non-invasively delivering an analgesic into a patient's systemic circulation comprises delivering the analgesic with a lozenge.

8. The method of claim 1, wherein the step of non-invasively delivering an analgesic into a patient's systemic circulation comprises delivering the analgesic with a lozenge attached to a handle.

9. The method of claim 1, wherein the step of non-invasively delivering an analgesic into a patient's systemic circulation comprises delivering the analgesic with an oromucosal patch.

10. The method of claim 1, wherein said unnecessary adverse side effect associated with excessively rapid increases in ASC comprises muscle rigidity.

11. The method of claim 1, further comprising the step of reducing an additional adverse side effect, wherein said additional adverse side effect comprises sedation.

12. The method of claim 1, further comprising reducing an additional adverse side effect, wherein said additional adverse side effect comprises dizziness.

13. The method of claim 1, further comprising reducing an additional adverse side effect, wherein said additional adverse side effect compromises nausea.

14. The method of claim 1, further comprising reducing an additional adverse side effect, wherein said additional adverse side effect compromises constipation.

15. The method of claim 1, further comprising reducing an additional adverse side effect, wherein said additional adverse side effect compromises respiratory depression.

16. The method of claim 1, further comprising reducing an additional adverse side effect, wherein said additional adverse side effect compromises vomiting.

17. The method of claim 1, further comprising reducing an additional adverse side effect, wherein said additional adverse side effect compromises somnolence.

18. The method of claim 1, wherein said analgesic is selected from a group consisting of: morphine, hydromorphone, levorphanol, heroin, fentanyl, sufentanil, remifentanil, alfentanil, a fentanyl derivative, methadone, buprenorphine, and oxycodone.

19. A drug formulation comprising:
a drug, said drug capable of conforming to an pharmacokinetic profile when administered to a patient's systemic circulation and, said pharmacokinetic profile providing a pharmacodynamic profile, said pharmacodynamic profile having an optimal onset of effect, optimal duration of effect, and an optimal rate of offset of effect; and
a carrier for administering said drug, said carrier providing user control over rate of absorption to maintain said optimal pharmacokinetic profile and said optimal pharmacodynamic profile.

20. The drug formulation of claim 19, wherein the drug is selected from the group comprising: morphine, hydromorphone, levorphanol, heroin, fentanyl, sufentanil, remifentanil, alfentanil, a fentanyl derivative, methadone, buprenorphine, and oxycodone.

21. The formulation of claim 19, wherein the drug is delivered oral transmucosally.

22. The formulation of claim 19, wherein the drug is delivered transdermally.

23. The formulation of claim 19, wherein the drug is delivered through the nasal mucosa.

24. The formulation of claim 19, wherein the carrier comprises a combination of pharmaceutical ingredients.

25. The formulation of claim 24, wherein the carrier further comprises a drug dosage form.

26. The formulation of claim 25, wherein the drug dosage form is an oral spray.

27. The formulation of claim 25, wherein the drug dosage form is a nasal spray.

28. The formulation of claim 25, wherein the drug dosage form is a lozenge.

29. The formulation of claim 25, wherein the drug dosage form is a lozenge attached to a handle.

30. The formulation of claim 25, wherein the drug dosage form is an oromucosal patch.

31. The formulation of claim 25, wherein the carrier provides user control over the rate of absorption by reducing absorption through secondary absorption routes.

32. The formulation of claim 19, wherein the carrier provides user control over the rate of absorption by reducing absorption into the systemic circulation through a primary absorption route.

33. The formulation of claim 19, wherein the optimal duration of effect is the time period from just after the breakthrough pain begins to just after the breakthrough pain ends.

34. A method for treating breakthrough pain of a breakthrough pain episode comprising:
administering an analgesic, said analgesic having a PK profile in which an initial increase in ASC occurs as the result of administering the analgesic at the beginning of a breakthrough pain episode, the rate of increase in ASC being adjusted to a patient's perception of increasing pain;
and in which a decrease in ASC absorption rate occurs as the result of reducing the amount of analgesic delivered before the pain is completely eliminated;
and in which ASC peaks at a safe ASC;
and in which a decreasing ASC occurs in part as a result of ending the administration of analgesic before the breakthrough pain episode has completely subsided;
and in which a rate of decrease in ASC during a period of time when the breakthrough pain subsides is not significantly affected by secondary absorption of the analgesic.

35. The method of claim 34, wherein said secondary absorption is delayed absorption of analgesic from a patient's GI tract.

36. The method of claim 34, wherein the rate of decrease in ASC is not affected by delayed absorption of analgesic from depot sites.

37. The method of claim 34, wherein administering an analgesic into a patient's systemic circulation comprises delivering the analgesic oral transmucosally.

38. The method of claim 27, wherein administering an analgesic into a patient's systemic circulation comprises delivering the analgesic transdermally.

39. The method of claim 34, wherein administering an analgesic into a patient's systemic circulation comprises delivering the analgesic through the nasal mucosa.

40. The method of claim 34, wherein administering an analgesic into a patient's systemic circulation comprises delivering the analgesic with an oral spray.

41. The method of claim 34, wherein administering an analgesic into a patient's systemic circulation comprises delivering the analgesic with a nasal spray.

42. The method of claim 34, wherein administering an analgesic into a patient's systemic circulation comprises delivering the analgesic with a lozenge.

43. The method of claim 34, wherein administering an analgesic into a patient's systemic circulation comprises delivering the analgesic with a lozenge attached to a handle.

44. The method of claim 34, wherein administering an analgesic into a patient's systemic circulation comprises delivering the analgesic with an oromucosal patch.

45. The method of claim 34, wherein said analgesic is selected from a group consisting of: morphine, hydromorphone, levorphanol, heroin, fentanyl, sufentanil, remifentanil, alfentanil, a fentanyl derivative, methadone, buprenorphine, and oxycodone.

46. A drug formulation for treating breakthrough pain comprising a drug, and a carrier, said carrier facilitating delivery of the drug to a patient's systemic circulation at a serum concentration level that corresponds to the minimum effective dose for a patient's specific pain level.

47. The drug formulation of claim 46, wherein said carrier facilitating delivery of the drug to the patient's systemic circulation is a dosage form selected from the group of: lozenge, lozenge attached to a handle, nasal spray, oral spray, and oromucosal patch or tablet.

48. The drug formulation of claim 46, wherein said carrier facilitates delivery of the drug to the patient's systemic circulation by providing sufficient concentrations of analgesic to meaningfully reduce the patient's pain.

49. The drug formulation of claim 46, wherein said carrier facilitates delivery of the drug to the patient's systemic circulation by delivering the drug in small portions over a period of time.

50. The drug formulation of claim 46, wherein said carrier facilitates delivery of the drug to the patient's systemic circulation by delivering the drug at a continuous, controllable rate.

51. The drug formulation of claim 46, wherein said carrier facilitates delivery of the drug to the patient's systemic circulation by a technique that allows a user to evaluate the progressive effect of the analgesic on the patient.

52. The drug formulation of claim 46, wherein said carrier facilitates delivery of the drug to the patient's systemic circulation by a technique that allows the user to adjust the absorption rate in response to a physiological effect(s).

53. The drug formulation of claim 46, wherein said carrier facilitates delivery of the drug to the patient's systemic circulation by a technique that allows the user to evaluate a patient's analgesia and terminate the administration to avoid overdosing.

54. The drug formulation of claim 46, wherein said carrier facilitates delivery of the drug to the patient's systemic circulation by administering the analgesic at an administration site that provides a relatively fast absorption rate and a relatively fast delivery to a patient's target tissue.

55. The drug formulation of claim 46, wherein said carrier facilitates delivery of the drug to the patient's systemic circulation by reducing absorption from secondary absorption routes.

56. The drug formulation of claim 46, wherein said carrier facilitates delivery of the drug to the patient's systemic circulation by enhancing absorption of the drug into tissues near the administration site.

57. The drug formulation of claim 46, wherein said carrier facilitates delivery of the drug to the patient's systemic circulation by terminating the administration prior to the end of the breakthrough pain episode.

58. The drug formulation of claim 46, wherein the drug is selected from the group of: morphine, hydromorphone, levorphanol, heroin, fentanyl, sufentanil, remifentanil, alfentanil, a fentanyl derivative, methadone, buprenorphine, and oxycodone.
